(19) United States
(12) Patent Application Publication
(10) Pub. No.: US 2013/0028814 A1
(43) Pub. Date: Jan. 31, 2013

(54) POROUS FILTER COLUMN AND REAGENT CARTRIDGE AND NUCLEIC ACID PURIFICATION KIT USING THE SAME

(71) Applicant: Toppan Printing Co., Ltd., Tokyo (JP)

(72) Inventor: Daisuke Numai, Tokyo (JP)

(73) Assignee: Toppan Printing Co., Ltd., Tokyo (JP)

(21) Appl. No.: 13/629,224

(22) Filed: Sep. 27, 2012

Related U.S. Application Data
(63) Continuation of application No. PCT/JP2011/050655, filed on Jan. 17, 2011.

(30) Foreign Application Priority Data
Mar. 31, 2010 (JP) 2010-082328

(51) Int. Cl. B01D 29/00; G01N 1/34

(52) U.S. Cl. 422/534; 210/435

(57) ABSTRACT

One embodiment of the present invention is a porous filter column as a cylindrical shape column having a bottom, including: an outlet at the bottom unit which holds a porous filter on the bottom unit; and, a hollow member which is placed on the porous filter, wherein the hollow member includes a hollow unit which is not in contact with an inner wall surface of the column and the porous filter, and a leg unit which is in contact with the inner wall surface of the column and the porous filter.

### CROSS REFERENCE TO RELATED APPLICATION

This application is a continuation of PCT International Application No. PCT/JP2011/050655, filed Jan. 17, 2011, the entire disclosure of which is incorporated herein by reference.

### BACKGROUND OF THE INVENTION

1. Field of the Invention

The present invention relates to a porous filter column used in filtration of liquid or extraction of a specific substance in liquid and, in particular, a porous filter holding method.

2. Background Art

Porous filters are widely used for research and industrial application as a tool for filtration of liquid or extraction of a specific substance in liquid. In such cases, the porous filter must be held within the column in which liquid is passed through. Normally, a method in which a porous filter is inserted and fixed by two members within the column and the like are used. Recently, the method has also been used for the extraction of nucleic acid of biological samples in the field of genetic engineering and genetic treatment.

As a method for nucleic acid extraction and collection, BOOM method is known. BOOM method is a nucleic acid separation and purification method which combines a chaotropic reagent, solid phase silica and the like using the fact that nucleic acid is adsorbed to a surface of silica in the presence of chaotropic ions. Separation and purification of nucleic acid are carried out by a method in which nucleic acid dissolved from a biological sample is adsorbed to porous silica, impurities are washed out using a cleaning solution, and the nucleic acid adsorbed to the silica is eluted by an eluent and is collected. In order to perform this method, cylindrical shape columns having a bottom where the porous filter is held at the bottom are used and these have outlets for liquid waste at the bottom and a plurality of samples are passed through the porous filter by pressuring means such as a pump or a centrifuge, thereby purified nucleic acid is collected eventually.

A column for nucleic acid extraction, together with a cylindrical shape outer container and a member for holding the porous filter, is mostly made of resin and the columns are often discarded after a single use without being used again in order to prevent contamination since they are used in precise experiments and measurements. Therefore, the cylindrical shape outer container and the porous filter are treated as one and the member for holding the porous filter is used.

As the member for holding the porous filter, an O-ring is commonly used, and in the cylindrical shape columns having a bottom where the porous filter is held, the O-ring is sometimes placed at the peripheral edge portion of the porous filter.

However, the portion of the filter in contact with the O-ring has poor filtration efficiency and has little movement of fluid even when pressurized since the filter area becomes narrow. Also, adsorption efficiency and cleaning efficiency are both poor as the porous filter when used in nucleic acid extraction since the solution is easy to remain as impurities in a corner or a gap sandwiched between the O-ring and the column, therefore, there have been problems of purity or yield of the sample being reduced.

As other methods for holding a porous filter, there is a method in which a porous filter is fixed by welding using adhesives, ultrasonic waves or a laser, or by integrating two molded products using insert molding in order to hold the porous filter within the cylindrical shape column as disclosed in JP4406344B.

In JP2008-86893A, a column with no adhesives and no filter pressing by an O-ring or the like is disclosed. In this method, a convex rib which protrudes within the column and bends the porous filter is provided, the end of the porous filter bends at the portion corresponding to the convex rib, and the porous filter is pressed by the reaction force from the bending and is fixed. In the column such as this, no member to hold the porous filter such as an O-ring is present, therefore, filtration efficiency is high and as a result, adsorption efficiency and cleaning efficiency are improved.

However, in the method disclosed in JP4406344B, costs for manufacturing the porous filter columns are increased since dedicated facilities are needed to manufacture the columns. Also in the method described in JP2008-86893A, the reaction force may be obtained if the porous filter is robust, however, if materials with sufficient strength for a porous filter cannot be used, this method may not be used, and since the range of choice for the porous filter is restricted, appropriate porous filters may not be selected when considering the combination of the sample to be adsorbed to the porous filter and the filter material.

Therefore, there has been a demand for filter columns, in which a filter is fixed within a column, purity and yield of the sample are improved by increasing filtration efficiency, and of which a cost is low.

The present invention has been made in view of the above described problems and an object of the invention is to provide means for fixing a filter within a column and to provide a filter column with high permeability to a solution, high movement of fluid, and also a low lost.

### SUMMARY OF THE INVENTION

The inventors have found technologies in which a hollow member instead of an O-ring holds a porous filter and a filter performance is maintained. Specifically, the invention is described as follows.

A first aspect of the present invention is a porous filter column including:

a cylindrical shape column having a bottom unit holding a porous filter;

an outlet at the bottom unit; and

a hollow member which is placed on the porous filter,

wherein the hollow member includes a hollow unit which is not in contact with an inner wall surface of the column and the porous filter, and a leg unit which is in contact with the inner wall surface of the column and the porous filter.

A second aspect of the present invention is the porous filter column according to the first aspect,

wherein the hollow member includes two or more leg units in contact with the inner wall surface of the column and the porous filter.

A third aspect of the present invention is the porous filter column according to the first aspect,

wherein a contact surface of the leg unit and the inner wall surface of the column have a circular arc shape.

A fourth aspect of the present invention is the porous filter column according to the third aspect,

wherein a curvature of the circular arc shape of the contact surface of the leg unit is the same as a curvature of the circular arc shape of the inner wall surface of the column.

A fifth aspect of the present invention is the porous filter column according to the fourth aspect,

wherein a central angle of the circular arc shape is from 10° to 30°.

A sixth aspect of the present invention is the porous filter column according to the first aspect,

wherein the hollow unit of the hollow member is circular.

A seventh aspect of the present invention is the porous filter column according to the first aspect,

wherein the porous filter has a nucleic acid adsorptivity.

A eighth aspect of the present invention is a reagent cartridge for accommodating a liquid to separate and purify a nucleic acid from an analyte, and dispensing the liquid using a dispensing tip, including:

an analyte accommodating unit which accommodates the analyte,

a liquid accommodating unit which accommodates the liquid,

a liquid waste accommodating unit which accommodates a liquid waste generated in the separation and purification, and

a porous filter column which purifies the nucleic acid of the analyte

wherein the porous filter column is the porous filter column according to the first aspect.

A ninth aspect of the present invention is a nucleic acid purification kit, including:

the reagent cartridge according to the eighth aspect, and

a dispensing tip accommodator for accommodating a plurality of dispensing tips.

By making the hollow unit not be contact with both the porous filter and the inner wall surface of the column, holding the porous filter without damaging the permeability to a solution and the movement of fluid as possible can be possible compared to atypical O-ring. Also, the porous filter is fixed by the leg unit therefore it is possible to prevent the positional deviation due to lifting of the filter and the like. Also, in the leg unit, by making the surface in contact with the inner wall surface of the column be a circular arc shape, the contact becomes closer and the fixing power of the hollow member itself to a column is strengthened.

### BRIEF DESCRIPTION OF THE DRAWINGS

FIG. 1 is a perspective view that shows a reagent cartridge according to one embodiment of the present invention.

FIG. 2 is a perspective view that shows a configuration of a reagent cartridge with a dispensing tip rack according to one embodiment of the present invention.

FIG. 3A is a top view of a porous filter column according to the present invention.

FIG. 3B is a cross-sectional view of a porous filter column according to the present invention.

FIG. 4 is a cross-sectional view of an outer container of a porous filter column according to the present invention.

FIG. 5A is a bird's eye view of a cross section partially cut in order to show a structure of a bottom surface unit of an outer container and a surrounding supporting unit of a porous filter column according to the present invention.

FIG. 5B is a bird's eye view of a cross section partially cut in order to show a structure of a bottom surface unit of an outer container and a surrounding supporting unit of a porous filter column according to the present invention.

FIG. 6A is a bottom view of a hollow member.

FIG. 6B is a lateral view of a hollow member.

FIG. 6C is a top view of a hollow member.

### DETAILED DESCRIPTION OF THE INVENTION

Hereinafter, the embodiments of the present invention will be described in detail with reference to the accompanying drawings. First of all, a nucleic acid purification kit configured of a reagent cartridge 100, in which a porous filter column 1 according to the present invention is stored and used for separation and purification of nucleic acid, and a dispensing tip rack 200 will be described.

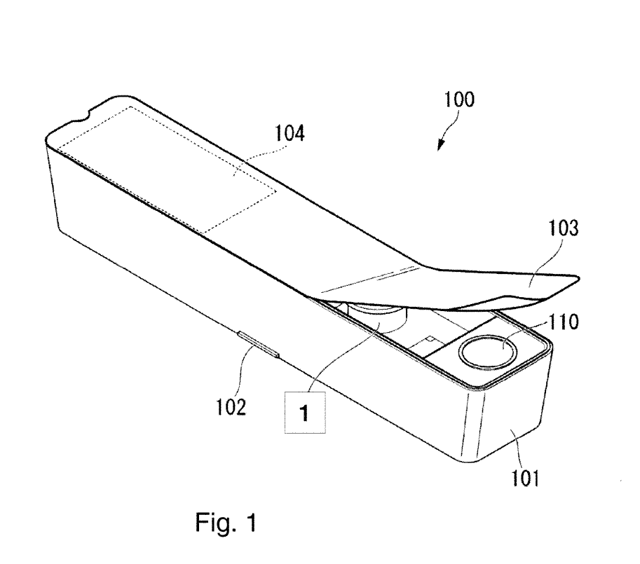

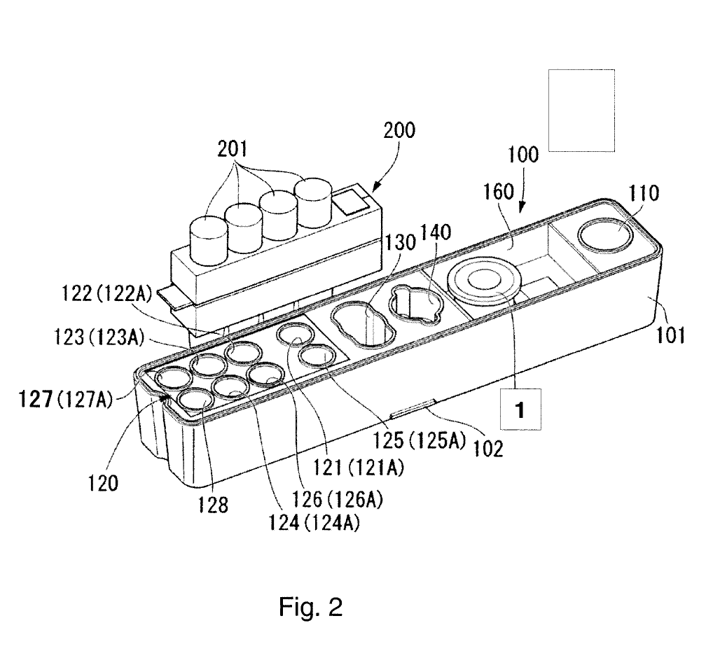

As shown in FIG. 1 and FIG. 2, the nucleic acid purification kit includes the reagent cartridge 100 in which a reagent to extract nucleic acid from an analyte and the like are accommodated and the dispensing tip rack 200 (dispensing tip accommodator) in which a dispensing tip 201 to dispense liquid is accommodated in plural numbers. In the present embodiments, the dispensing tip rack 200 includes a plurality of the dispensing tips 201 of same shape and same size, liquid accommodated in the reagent cartridge 100 is dispensed to or stirred by any of a plurality of the dispensing tips 201 so that cross-contamination between liquids does not occur by the dispensing tips 201. In addition, the dispensing tip rack 200 is also a container for collecting the dispensing tips 201 after use, therefore, the dispensing tips 201 as infectious waste may be discarded as contained in the dispensing tip rack 200 after the use of the dispensing tips 201 is completed.

FIG. 1 is a perspective view that shows a reagent cartridge 100. The reagent cartridge 100 has a main body 101 formed approximately in a box shape and a claw unit 102 formed protruding from the outer surface of the main body 101. The claw unit 102, for example, is made to be able to engage with a part of the nucleic acid analyzing apparatus so as to avoid tipping of the reagent cartridge 100 when the reagent cartridge 100 is set in the nucleic acid analyzing apparatus or the like.

On part of the outer surface of the main body 101, a thin-film-shaped sealing film 103 which becomes detached when used is attached. An opening of the main body 101 is sealed with the sealing film 103 and prevents falling out of the porous filter column 1 described later and the like disposed inside the main body 101 from the main body 101 and prevents incorporation of foreign substances such as dust into the inside of the main body 101.

FIG. 2 is a perspective view that shows the reagent cartridge 100 and the dispensing tip rack 200 without the sealing film 103. Inside the main body 101, a sample well (analyte accommodating unit) 110 in which an analyte such as a biological sample and the like is introduced, a reagent well unit 120 in which a reagent for extracting nucleic acid from the analyte is accommodated, a liquid waste well (liquid waste accommodating unit) 130 in which unnecessary solution separated in a step of extracting nucleic acid from the analyte is discarded, and a collection well 140 in which nucleic acid extracted from the analyte is collected are formed as one unit. Also, in the reagent cartridge 100, a holding unit 160 in which the porous filter column 1 of the present invention is accommodated is formed as one unit.

The holding unit 160 is at an initial position where the porous filter column 1 of the present invention is accommodated in the reagent cartridge 100. Also at the bottom unit of the holding unit 160, an absorber, not shown, which absorbs liquid may be provided. This absorber is made to be in contact with the outer surface of an outlet 17 side of the porous filter column 1 when the porous filter column 1 is accommodated in the holding unit 160. For this reason, for example, if cleaning solution is adhered on the outer surface of the outlet 17 when the cleaning solution is supplied within the porous filter column 1, the cleaning solution may be removed by absorbing the cleaning solution to the absorber.

The reagent well unit 120 has a plurality of reagent wells (reagent accommodating units) 121, 122, 123, 124, 125 and 126, an oil well (oil accommodating unit) 127, and an oil removing unit (liquid removing unit) 128. Also, in the reagent well unit 120, openings of a plurality of the reagent wells 121, 122, 123, 124, 125 and 126, and the oil well 127 are sealed with a sealing film 104. The sealing film 104 preferably has a configuration which suppresses permeation of gas and can be torn by stabbing of the dispensing tip 201 and, for example, a thin film made of metal, a plastic film or the like may be used.

In the reagent wells 121 to 126, a dissolving solution 121A which dissolves biological substances such as cell membrane, a dissolving solution 122A which dissolves biological substances such as cytoplasm not completely dissolved in the above dissolving solution 121A and causing clogging in a carrier, cleaning solutions 123A and 124A for washing out unwanted substances other than nucleic acid adsorbed to the carrier, an eluent 125A eluting nucleic acid from the carrier, and a diluting liquid 126A for adjusting a nucleic acid concentration in eluent are individually accommodated in each reagent well.

In the oil well 127, for example, a well-known oil 127A used for overlaying to a reaction solution in a PCR reaction is accommodated. As the oil 127A, for example, mineral oil, silicon oil or the like may be suitably employed.

As shown in FIG. 2, a liquid waste well 130 has a concave unit formed according to an outer diameter shape of the porous filter column 1. The inner diameter of this concave unit is larger than the outer diameter of a side surface unit 12, therefore, from the outlet 17 to a protrusion 15 of the porous filter column 1 is inserted within the well. However, the inner diameter of the concave unit is smaller than the outer diameter of the protrusion 15 described later formed in the side surface unit 12 of the porous filter column 1, therefore, this protrusion 15 and the opening unit of the liquid waste well and the collection well are engaged and the outlet 17 may hold the porous filter column 1 at a height not to be in contact with liquid waste in the liquid waste well. Also, this concave unit has a shape capable of holding the porous filter column 1 since it has the inner diameter shape matching with the outer diameter shape of the porous filter column 1, therefore, the porous filter column 1 does not tip within the reagent cartridge 100 under the condition of the porous filter column 1 being installed in the liquid waste well 130.

The collection well 140 can also hold the porous filter column 1 similar to the liquid waste well 130. The bottom unit of the collection well 140 has a container shape so that nucleic acid solution eluted by the eluent 125A from the carrier of the porous filter column 1 can be stored.

The liquid waste well 130 and the collection well 140 are provided at a neighboring location within the reagent cartridge 100. This is for the flow of the porous filter column 1 to be decreased when the porous filter column 1 is moved to the collection well 140 after cleaning of the porous filter column 1 in the liquid waste well 130 is carried out. As a result, the possibility of the porous filter column 1 which passes through the reagent cartridge 100 contaminating the reagent cartridge 100 and the like may be reduced.

Next, the porous filter column 1 according to the present invention is described.

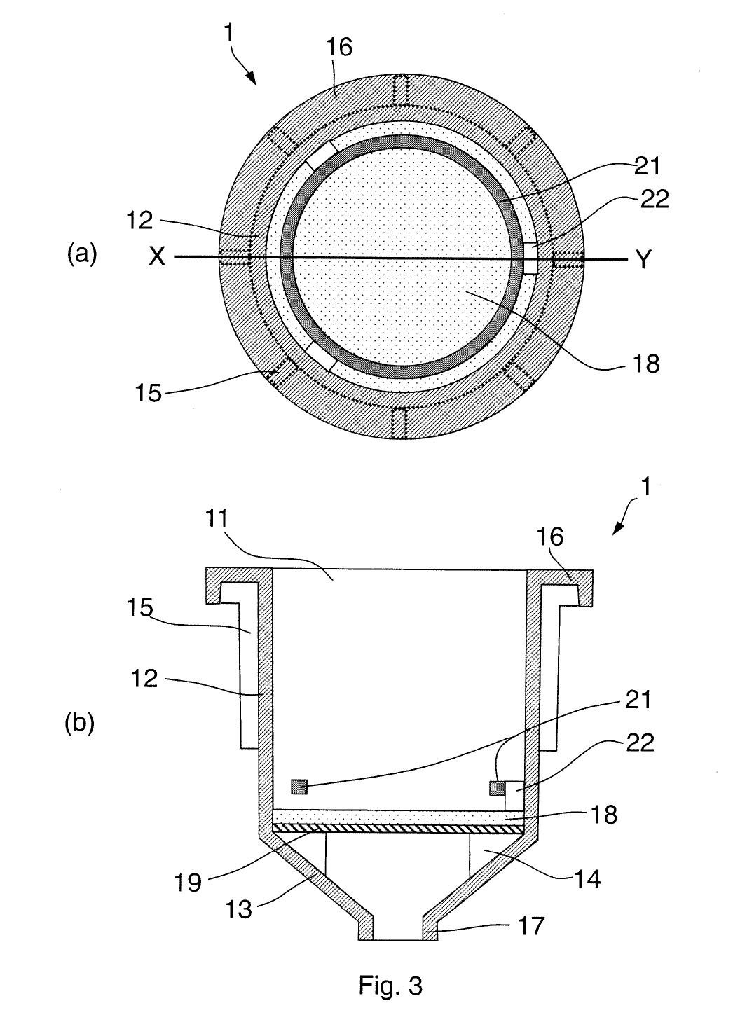

FIG. 3a is a top view of a porous filter column 1 according to the present invention and FIG. 3b is a cross-sectional view between X-Y in FIG. 3a of a porous filter column 1 according to the present invention. The porous filter column 1 includes a circular shape porous filter 18, a circular shape holding member 19, a cylindrical shape outer container 10 in which the circular shape porous filter 18 and the circular shape holding member 19 are stored, and a hollow member 20 placed on the circular shape porous filter 18.

The porous filter column 1 is configured as the combination of the outer container 10, the porous filter 18 and the hollow member 20 as shown in FIG. 3. Test solutions such as a sample solution or a cleaning solution described later are dispensed from an opening unit 11 located at the top of the outer container 10, and then, by introducing pressurized air, the test solution is adsorbed to the porous filter 18 or is passed through and filtered, and is discharged from the outlet 17 or collected in a separate container.

Next, the outer container 10, the porous filter 18, the holding member 19, and the hollow member 20 are described in detail.

### <Outer Container 10>

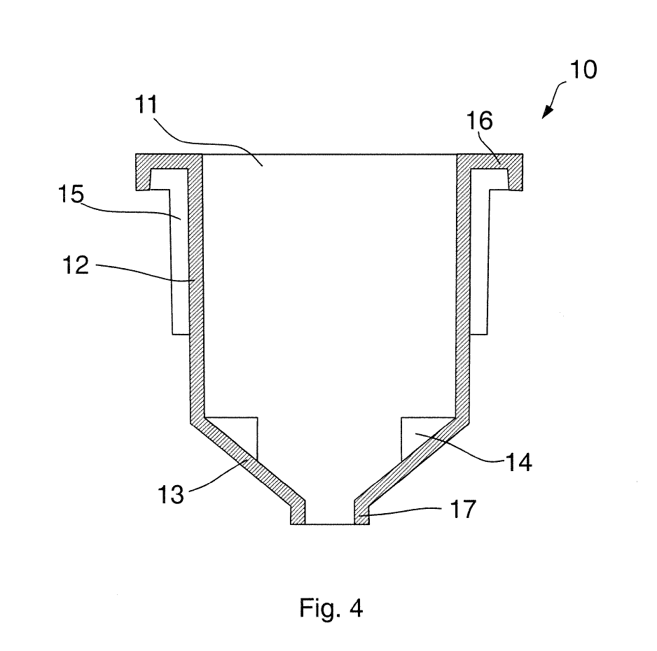

FIG. 4 is a cross-sectional view of an outer container 10 and the outer container 10 includes at least an opening unit 11 at the top, a cylindrical shape side surface unit 12, a funnel-shaped bottom surface unit 13 and the nozzle-shaped outlet 17 protruding at the center of the bottom surface unit 13, and the outer container 10 is configured by molding these as one unit. Also, a flange unit 16 may be formed near the opening unit 11 and the protrusion 15 may be formed near the side surface unit 12. The shape of the outer container 10 is a tubular shape with the opening unit 11 at the top and the outlet 17 at the bottom both open for through passage, therefore, the dissolving solution in which the analyte is dissolved or the cleaning solution, eluent and the like are supplied from the opening unit 11 at the top. These liquids passing through the porous filter 18 and the holding member 19 are able to be discharged from the outlet 17.

The bottom surface unit 13 of the outer container 10 is formed in a funnel shape with the inner diameter becoming smaller toward the outlet 17 side from the porous filter 18 side. The top of the bottom surface unit 13 is formed horizontally since the holding member 19 is placed on the bottom surface unit 13 to be horizontal. Also, the bottom surface unit 13 descends as the porous filter 18 side faces the outlet 17 side, therefore, sample liquid injected from the opening unit 11 flows down the descended bottom surface unit 13 and is easily discharged from the outlet 17. Also, the nozzle-shaped outlet 17 is formed to protrude to a bottom direction at the center of the bottom surface unit 13.

Between the bottom surface unit 13 and the holding member 19 of the outer container 10, a supporting unit 14 as one unit with the outer container 10 may be formed for the purpose of supporting and prevention of deformation of the holding member 19 and the porous filter 18. Formation of a gap between the porous filter 18 on the holding member 19 and the bottom surface unit 13 is prevented since the supporting unit 14 is in contact with the holding member 19. In addition, the surface contacted with the holding member 19 of the supporting unit 14 is formed horizontally in order to support the filtering surface of the porous filter 18 to be horizontally uniform.

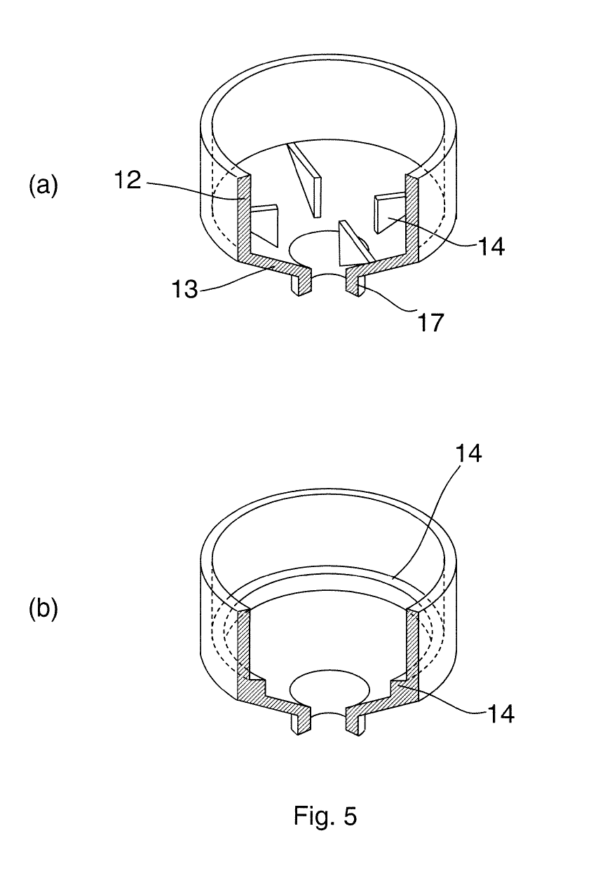

FIG. 5 is a bird's eye view of a cross section partially cut in order to show a structure of a bottom surface unit 13 of the outer container 10 and a surrounding supporting unit 14. As the shape of the supporting unit 14, for example as in FIG. 5a, a plate shape of four sheets disposed in a radial shape centering around the outlet 17 in the bottom surface unit 13 and protruding from the descended bottom surface unit 13 may be included, and in this case, since even the center of the porous filter 18 can be supported since deformation of the porous filter 18 is difficult to occur when pressurized, a porous filter 18 with low strength may be used. Also, when the same shape of the supporting unit 14 as in FIG. 5a is provided, filtration efficiency decreases since the supporting unit 14 blocks the filtering surface of the porous filter 18 and the filtering surface becomes narrower. However, when viscosity or concentration of the sample liquid is high and high filtration efficiency is necessary, a ring-shaped supporting unit 14 one stage higher than the bottom surface unit 13 may be formed, as shown in FIG. 5b, in order to widen the filtering surface of the porous filter 18.

In an inner side of the outer container 10, the porous filter 18 and the holding member 19 are held by being placed in the supporting unit 14. The inner diameter of cylindrical shape of the side surface unit 12 is preferably the same as the outer diameter of the porous filter 18. As a result, formation of a gap between the side surface unit 12 and the porous filter 18 may be prevented. Also, the inner diameter of the side surface unit 12 is formed as a tapered surface in which the diameter decreases from the opening unit 11 at the top toward the bottom surface unit 13 and the inner diameter of the side surface unit 12 and the outer diameter of the porous filter 18 may be matched at the top of the bottom surface unit 13 where the diameter of the tapered surface is minimized.

Around the opening unit 11 at the top of the outer container 10, the flange unit 16 may be formed in order to move the porous filter column 1. When a cleaning step and a collecting step of the nucleic acid sample is performed, each step is performed after moving the porous filter column 1 from the initial state in which it is stored in the reagent cartridge to the liquid waste well or the collection well, therefore, moving by a porous filter column moving means becomes possible by forming the flange unit 16 for moving near the opening unit 11 at the top of the porous filter column 1.

Also, on the periphery of the outer container 10, the protrusion 15 may be formed outside the side surface unit 12 so as for the outlet 17 not to contact the liquid waste well of the reagent cartridge. This protrusion 15 is larger in diameter than an inner diameter of the liquid waste well and the collection well, therefore, this protrusion 15 and the opening unit of the liquid waste well and the collection well are engaged and the porous filter column 1 does not tip and is held. Also by setting the location of the side surface unit 12 in which this protrusion 15 is formed at a height for the outlet 17 not to be in contact with liquid waste in the liquid waste well when the protrusion 15 and the opening unit of the liquid waste well and the collection well are engaged, the tip of the outlet 17 or the surroundings is not contaminated by liquid waste in the cleaning step in the liquid waste well and, in the collection step afterward, the possibility of the waste being incorporated into the eluted sample may be reduced.

This protrusion 15 is preferably formed as one unit with the outer container 10. Also, as the shape of this protrusion 15, a shape which can engage with the opening unit of the liquid waste well and the collection well so that the porous filter column 1 does not tip and is held may be used and a protrusion with a shape of a plurality of plates or with a shape of rod, or a ring-shaped protrusion 15 which covers a periphery of the outer container 10 may also be used. Also, the circumference of the circle made from the tips of a plurality of protrusions or the diameter of the ring-shaped protrusion 15 is preferably larger than the inner diameter of the liquid waste well and the collection well since this protrusion 15 and the opening unit of the liquid waste well and the collection well are engaged.

The materials forming the outer container 10 are not particularly limited as long as they do not dissolve in solvents used in sample solutions and do not have an influence on the samples or reagents in solution, however, particularly, if resin materials including any of polypropylene, polycarbonate and acryl are used, satisfactory visible light transmittance is secured, therefore, status of the solutions may be checked. As the polypropylene, homopolypropylene or a random copolymer of polypropylene and polyethylene may be used. Also, as the acryl, polymethyl methacrylate or a copolymer with a monomer such as methyl methacrylate and other mathacrylates, acrylates, and styrene may be used. Also, when these resin materials are used, heat resistance or strength of the tip may be secured. As a manufacturing method of the outer container 10, various resin molding methods such as an injection molding or a vacuum molding, or machine cutting may be used.

### <Porous Filter 18>

The porous filter 18 uses materials having hydrophilic groups on the surface to which biological samples are chemically adsorbed, and preferably, is formed in a porous film shape with a large surface area in order for the sample solution to pass through the inside and for the sample to be adsorbed efficiently. Also, it has a configuration to adsorb and support nucleic acid when cleaning by the cleaning solution and to weaken the adsorptive power of the nucleic acid and to detach when collecting by a collecting liquid. The porous material in the present invention includes those in which fibrous materials such as glass wool are overlaid.

As the shape of the porous filter 18, an outer diameter shape in which no gap with the outer container 10 is formed when installed horizontally within the outer container 10 may be used, and if the outer container 10 is a cylindrical shape, a circular shape having the same outer diameter as the inner diameter of the outer container 10 is preferable. Also, a film thickness of the filter varies depending on the filter materials used since adsorbability of the sample changes depending on the types of hydrophilic group, surface area of porous materials, types of sample which need to be adsorbed or the like, and setting the film thickness just sufficient to adsorb the sample required for analysis and the like is preferable.

Also, the porous filter 18 installed within the outer container 10 may be used as one sheet, however, a plurality of sheets may be used, and materials of the porous filter 18 when a plurality of sheets are used may be the same as or different from each other.

Materials of the filter are not particularly limited as long as biological substances such as nucleic acid can be adsorbed in the presence of organic materials, however, using a material having a hydrophilic group made to be porous or a porous material to which a hydrophilic group is introduced is preferable. As inorganic materials having a hydrophilic group, silica, a silica derivative in which a hydrophilic group is introduced to silica, diatomite, alumina or the like may be included. Also, as organic materials having a hydrophilic group, polyhydroxyethyl acrylate, polyhydroxyethyl methacrylate, polyvinyl alcohol, polyvinyl pyrrolidone, polyacrylic acid, polymethacrylic acid, polyoxyethylene, acetyl cellulose, a mixture of acetyl celluloses with different acetyl values, an organic material with a polysaccharide structure or the like may be used.

Also, a material having a hydrophilic group coated on the surface of a material having no hydrophilic group such as glass or ceramics may be used, and as the material used for coating, polyhydroxyethyl acrylate, polyhydroxyethyl methacrylate, and salts thereof, polyvinyl alcohol, polyvinyl pyrrolidone, polyacrylic acid, polymethacrylic acid, and salts thereof, or a polymer of organic material such as polyoxyethylene, acetyl cellulose, a mixture of acetyl cellulose with different acetyl values are preferable.

Here, the hydrophilic group indicates a polar group which can have an interaction with water and all groups involved in adsorption of biological substances such as nucleic acid and the like are relevant. The hydrophilic group such as this includes a polar group having an interaction with water which can adsorb nucleic acid and, for example, a hydroxyl group, a carboxyl group, a cyano group, an oxyethylene group, an amino group, or a group modified with these hydrophilic groups for the purpose of controlling hydrophilicity may be included.

### <Holding Member 19>

The porous filter 18 is pressurized by a pressurizing means when cleaning and filtering, therefore, when the porous filter 18 of low strength is used, the porous filter 18 bends and a gap in which solution passes through is formed between the outer container 10 and the porous filter 18, and a risk of solution leaking from this part exists. However, the bending of the porous filter 18 is prevented by disposing the holding member 19 of high rigidity, therefore, pressurization may be carried out even when a porous filter 18 of low strength is used. As a result, the rigidity of the holding member 19 is higher than that of the porous filter 18 and deformation of the porous filter 18 in the outer container 10 is suppressed by the holding member 19.

Also, preferably, the holding member 19 is formed by a material which, at least, has low adsorbability with regard to nucleic acid and does not inhibit an extraction reaction of nucleic acid from an analyte.

The holding member 19 is preferably a member in a filter shape from the viewpoint that liquid produced by sintering resin particles can pass through, however, it is not limited to this, and may be a member which does not dissolve in solvents used in cleaning and the like, of which rigidity is not reduced by the solvents, in which substances influencing a sample, a reagent or the like are not eluted, and which has a hole so that target solutions, impurities or the like can pass through. In producing the holding member 19, the same resin materials may be used as those of outer container 10, however, the material is not particularly limited as long as it is formed to be porous so that solutions can readily pass through toward the filter.

### <Hollow Member 20>

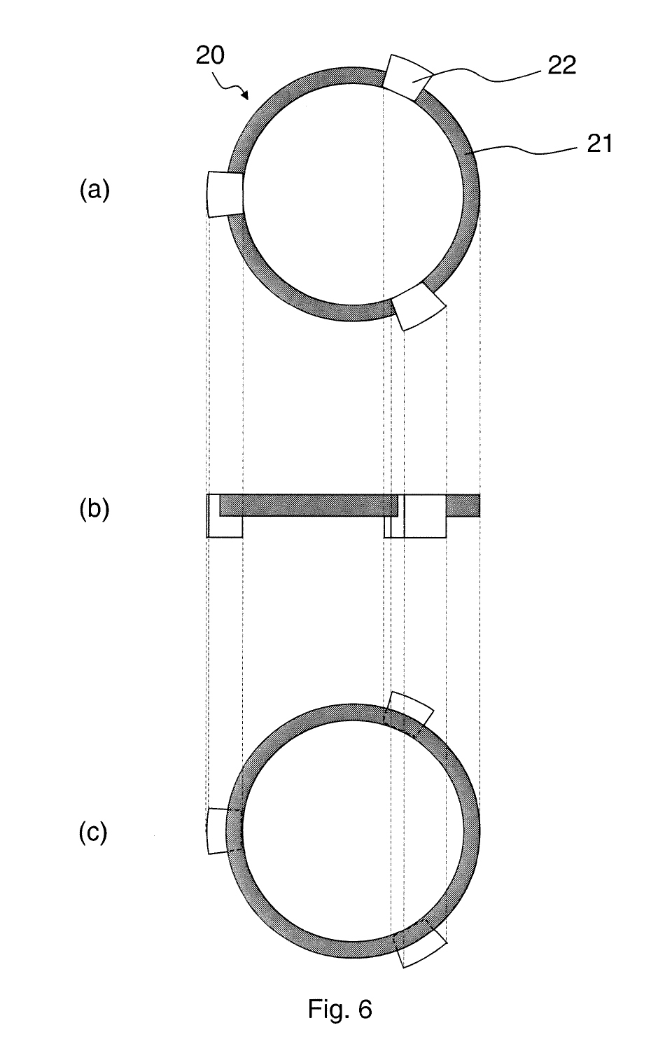

The hollow member 20, as show in FIG. 6, is configured of a hollow unit 21 and a leg unit 22. FIG. 6 is one example of the hollow member 20 and represents the hollow unit 21 as circular shape and three leg units 22. FIG. 6a is a diagram that shows the hollow member seen from immediately below, FIG. 6b is a diagram that shows the hollow member seen from the immediate side, and FIG. 6c is a diagram that shows the hollow member seen from immediately above.

The hollow unit 21 is designed to be in non-contact with the inner wall surface of the outer container 10 and the porous filter 18, and compared to an O-ring 30 which is a conventional full-scale contact type holding member, a solution is easily filtered since the contact surface between the porous filter 18 and the outer container 10 is small, and it is difficult for residual liquid of the solution to be generated. Also, the leg unit 22 responsible for the fixation to the outer container 10 by preventing the lifting of the hollow member 20 itself is extended from the hollow unit 21.

The leg unit 22, the outer container 10, and the porous filter 18 are in contact and lifting of the hollow member 20 itself is prevented by the friction between the inner wall surface of the outer container 10 and the side surface of the leg unit 22, therefore, lifting of the porous filter 18 pressed to the bottom of the leg unit 22 is prevented. For this reason, the outer diameter including the side surface of the leg unit of the hollow member 20 and the inner diameter of the outer container 10 are preferably the same in order for the leg unit 22 to contact with the inner wall surface of the outer container 10.

Also, filtration efficiency of the solution is enhanced compared to a case using a conventional O-ring since the hollow unit 21 is supported so that the hollow unit 21 and the porous filter 18 are not contacted by the leg unit 22 and there is a space for solution to pass through between the hollow unit 21 the porous filter 18. Also, even when a porous filter 18 of low strength is used, deformation of the porous filter 18 by pressurization when cleaning or extracting may be suppressed.

As the shape of the hollow unit 21, all polygonal shapes and circular shapes may be used, however, circular shapes are preferable from the viewpoint that designing of the extended leg unit is easy and residual liquid of the solution is suppressed as much as possible. Also, the outer diameter of the hollow unit 21 is preferably smaller than the inner diameter of the inner wall surface of the outer container 10 so that the contact between the hollow member 20 and the inner wall surface of the outer container 10 is reduced. As a result, a gap in which solution can pass through is formed between the hollow unit 21 and the inner wall surface of the outer container 10 and it is difficult for residual liquid of the solution to be generated. In the case of polygonal shapes, it is preferable that several vertices be made to be the leg units 22 and only the leg units 22 which are vertices be in contact with the inner wall surface of the outer container 10. Also, when the hollow unit 21 has a polygonal shape and is in contact with the inner wall surface of the outer container 10 only by vertices of the polygonal shape, it is difficult for residual liquid of the solution to be generated since the contact area is small, therefore, it may be considered that the hollow unit 21 and the inner wall surface of the outer container 10 are not in contact.

As the leg unit 22, if at least two or more leg units 22 are formed in the hollow unit 21, holding the outer container 10 by the porous filter 18 and the hollow member 20 itself becomes possible. However, three leg units are preferable from the viewpoint that the contact with the porous filter 18 is suppressed to a minimum level for higher filtration efficiency and the leg unit is stable and independent. As the height of leg unit 22, a height higher than a thickness of the hollow unit 21 and a height which can support the hollow unit 21 so that the hollow unit 21 and the porous filter 18 are not in contact are appropriate.

As the shape of the leg unit 22, a cross-sectional shape of the same horizontal direction as the horizontal surface on which the leg unit 22 is placed may be any shape such as a circular shape, a semi-circular shape, a trapezoidal shape, a square shape, or a rectangle shape. Also, the shape of the side surface of the leg unit 22 is preferably a circular arc shape in which the contact area with the inner wall surface of the outer container 10 is large, and is preferably a cross-sectional shape in which the side surface is a circular arc shape since the friction in which the side surface of the leg unit 22 and the inner wall surface of the outer container are contacted becomes large, therefore, the lifting prevention effect is increased by enlarging the area where the inner wall surface of the outer container 10 and the side surface of the leg unit 22 are contacted, as shown in FIG. 6. Particularly, it is most preferable that the curvature of the inner wall surface of the outer container 10 and the curvature of the side surface of the leg unit 22 are made to be the same in order to maximize the contact area between the side surface of the leg unit 22 and the inner wall surface of the outer container 10.

Also, the leg unit 22 may have a tapered shape in which the diameter gradually decreases toward the porous filter 18. In this case, filtration efficiency may be enhanced since the bottom area of the leg unit is smaller. For this reason, the cross-sectional shape of the leg unit 22 on a surface where the leg unit 22 crosses at right angles with the horizontal surface on which the leg unit 22 is placed is made to be a shape such as semi-circular diameter, circular shape, semi-circular shape, or trapezoidal shape and the surface with which an area in contact with the porous filter 18 is smallest is made to be the bottom unit of the leg unit 22 among these shapes.

Also, as the circular arc of the side surface shape of the leg unit 22, a circular arc of which a central angle is 10° to 30° is preferable. If the central angle is smaller than 10°, the contact area between the inner wall surface of the outer container 10 and the side surface of the leg unit 22 is small, therefore, the lifting prevention effect of the porous filter 18 decreases. Also, if the central angle is larger than 30°, the contact area between the bottom surface of the leg unit 22 and the porous filter 18 becomes large, therefore, filtration efficiency of the porous filter 18 decreases. In addition, it is easy for residual liquid of the solution to be generated since the contact area between the inner wall surface of the outer container 10 and the side surface of the leg unit 22 becomes large.

The materials which form the hollow member 20 are not particularly limited as long as they do not dissolve in solvents used in cleaning and the like or do not have an influence on the samples, reagents or the like, however, resin materials including any of polypropylene, polycarbonate and acryl, and molding methods are preferable similar to the outer container 10.

As inserting methods of the holding member 19, the porous filter 18, and the hollow member 20 into the outer container 10, well-known assembly robots or manufacturing methods may be used, and a method in which the holding member 19, the porous filter 18, and the hollow member 20 may be stacked in this sequence on the outer container 10 so as for each of them to be horizontal may be particularly suitably used. As a result, the porous filter column 1 of the present invention may be manufactured at low cost.

The porous filter column 1 of the present invention will be described focusing on its operation based on examples of nucleic acid separation and purification by a nucleic acid purification kit configured of the reagent cartridge 100 equipped with the porous filter column 1 relating to the present invention with the configuration described above and the dispensing tip rack 200.

First of all, the sealing film 103 of the reagent cartridge 100 shown in FIG. 1 is detached manually by a user. Subsequently, for example, a whole blood sample is injected to the sample well 110 of the reagent cartridge 100 manually by the user.

Subsequently, various reagents stored in the reagent wells 121 to 126 are dispensed and mixed according to predetermined orders by a dispense transport mechanism of the automated analyzing apparatus. As a result, cells in the whole blood sample supplied to the sample well 110 dissolve and a cell-dissolved solution may be obtained. When liquid from the reagent wells 121 to 126 is absorbed to the inside of the dispensing tip 201, the tip of the dispensing tip 201 is inserted to the sealing film 104 which seals the reagent wells 121 to 126. Then, a through hole is formed on the sealing film 104 and various reagents inside the reagent wells 121 to 126 may be absorbed by the dispensing tip 201.

Above these, the porous filter column 1 is transported to the liquid waste well 130 since liquid waste needs to be collected. The cell-dissolved solution is supplied to the porous filter column 1. The speed of the liquid passing through the porous filter 18 may be increased by the porous filter column 1 sending gas from the opening unit 11 and pressurizing the inside of the porous filter column. Then the cell-dissolved solution passes through the porous filter 18 and nucleic acid is adsorbed to the porous filter 18. After that, the porous filter 18 is cleaned using the dissolving solution 122A which dissolves biological substances such as cytoplasm not completely dissolved in the dissolving solution 121A causing clogging in a carrier.

Also, cleaning solutions 123A and 124A are supplied to the porous filter 18 and the porous filter 18 is cleaned with cleaning solutions 123A and 124A. After that, the porous filter column 1 is transported to the collection well 140 and the eluent 125A is supplied to the porous filter 18. As a result, nucleic acid adsorbed to the porous filter 18 is eluted in the eluent 125A and nucleic acid solution containing nucleic acid is collected in the collection well 140.

Also, the diluting liquid 126A is mixed with the eluent 125A in which collected nucleic acid is collected, and the sample preparation is completed. Then, separation and purification of nucleic acid by the nucleic acid purification kit equipped with the porous filter column 1 of the present invention is completed.

When sending gas to the porous filter column 1 and pressurizing, problems such as an end of the porous filter 18 being lifted and the like and if a conventional O-ring which suppresses lifting is used, a reservoir of liquid is generated between the O-ring and the outer container of the column, therefore, problems such as loss of the sample in the cleaning step or purity decrease of the sample due to incorporation of impurities exist. However, in the present invention, the hollow member 20 suppresses lifting of the porous filter 18 and no reservoir of liquid is generated between the hollow member 20 and the outer container 10, therefore, loss of the sample in the cleaning step or incorporation of impurities due to a reservoir of liquid is reduced and the yield and purity of the sample may be increased.

Hereinafter, one example of the embodiments of the present invention will be described with reference to examples, however, the present invention is not limited to these.

### EXAMPLES

### Nucleic Acid Extraction from Whole Blood

Results of nucleic acid extraction from whole blood using the porous filter column 1 described above are shown below.

### Example 1

### <1> Preparation of Porous Filter Column 1

In the outer container 10, a column with the same configuration shown in FIG. 3 was prepared by loading the holding member 19, the porous filter 18, and the hollow member 20 on the bottom surface unit 13 in this sequence. A molding product of polypropylene with an inner diameter of 13 mm was used as the outer container 10. As the hollow member 20, the same shape as that shown in the FIG. 6a to FIG. 6c was molded with polypropylene. The hollow unit 21 has a circular shape with an outer diameter of 11.9 mm, an inner diameter of 10.7 mm and a thickness of 1 mm, and a circular arc shape with an outer diameter of 13.1 mm, an inner diameter of 10.9 mm and a thickness of 2 mm with three leg units, and the central angle was 10°. As the holding member 19, a filter with a diameter of 13 mm and a thickness of 1 mm prepared by sintering propylene particles was used. As the porous filter 18, a glass fiber filter with a diameter of 13 mm, an average pore diameter of 1 μm and a thickness of 700 μm was used.

### <2> Preparation of Dissolving solution and Cleaning Solution

Dissolving solution (including 4M guanidine hydrochloride, 10 v/v % TritonX-100, 50 mM Tris-HCl, 10 mM EDTA) and cleaning solution (including 3 mM Tris-HCl, 0.3 mM EDTA, 30 mM NaCl, 70 v/v % ethanol) were prepared.

### <3> Nucleic Acid Extraction Operation

100 μl, of whole blood and 500 μL of dissolving solution prepared from <2> were mixed and stirred at 55° C. for 2 minutes, the mixture was dispensed from the opening unit 11 at the top of the porous filter column 1 prepared from <1>, was made to contact with the porous filter 18, and was incubated for one minute. Next, the whole blood dissolving solution was discharged by introducing pressurized air using a pump.

Hereinafter, dispensing and discharging operation with 600 μL of dissolving solution, 650 μL of cleaning solution prepared from <2>, and 300 μL of pure water was also carried out in a similar manner (repeating twice in case of cleaning solution). At the end, 350 μL of collecting liquid (pure water) heated to 55° C. was dispensed and nucleic acid was eluted. The above operation was repeated twice for 3 types of column samples each.

### <4> Measurement of Nucleic Acid Yield and Purity

For each nucleic acid solution obtained from <3>, yield was measured by a PicoGreen quantitation method and purity was measured by an absorbance determination method (A₂₆₀/A₂₈₀ and A₂₆₀/A₂₃₀), respectively.

### Comparative Example 1

A porous filter column was prepared in the same manner as that of Example 1 except that an O-ring with an outer diameter of 13.2 mm, an inner diameter of 10 mm and a thickness of 1.5 mm molded with polypropylene was loaded instead of the hollow member 20, and the <2> preparation of dissolving solution and cleaning solution <3> nucleic acid extraction operation <4> measurement of nucleic acid yield and purity were performed in the same manner as those of Example 1.

### Reference Example

A porous filter column was prepared in the same manner as that of Example 1 except that a column, in which the hollow member 20 was not loaded and only the holding member 19 and the porous filter 18 were loaded, was prepared, and the <2> preparation of dissolving solution and cleaning solution <3> nucleic acid extraction operation <4> measurement of nucleic acid yield and purity were performed in the same manner as those of Example 1.

Next, measurement results of Example 1, Comparative Example 1 and Reference Example are shown in Table 1. Each measurement value is an average value of two trials. Also, Nucleic Acid Purity 1 (A₂₆₀/A₂₈₀) is an indicator of protein incorporated into nucleic acid and Nucleic Acid Purity 2 (A₂₆₀/A₂₃₀) is an indicator of dissolving solution component incorporated into nucleic acid.

TABLE 1

| | Nucleic Acid | Nucleic Acid | Nucleic Acid |
|---|---|---|---|
| | yield [μg] | Purity 1 | Purity 2 |
| Example 1 | 0.80 | 1.58 | 0.31 |
| Comparative | 0.62 | 0.84 | 0.14 |
| Example 1 | | | |
| Reference | 0.88 | 1.43 | 0.43 |
| Example | | | |

As shown in Table 1, comparing to reference example (only the porous filter 18 and the holding member 19), yield and purity are slightly decreased, however, almost the same values are maintained in Example 1 in contrast to significant decreases of values in Comparative Example 1 (typical O-ring). Therefore, it may be considered that equivalent nucleic acid adsorption efficiency and cleaning efficiency are being maintained in Example 1 comparing to those in reference example. These results show that, in the porous filter column of the present invention, the porous filter 18 is held within the column, and also, original performance of the filter with regard to permeability of solution and movement of fluid is not impaired as possible.

The technical scope of the present invention is not limited to each embodiment described above and includes additions of various modifications to each embodiment described above without departing from the spirit or scope of the present invention. That is, specific materials, configurations or the like included in each embodiment are no more than one example of the present invention and appropriate modifications are possible.

What is claimed is:

1. A porous filter column comprising:
a cylindrical shape column having a bottom unit holding a porous filter;
an outlet at the bottom unit; and
a hollow member which is placed on the porous filter,
wherein the hollow member includes a hollow unit which is not in contact with an inner wall surface of the column and the porous filter, and a leg unit which is in contact with the inner wall surface of the column and the porous filter.

2. The porous filter column according to claim 1,
wherein the hollow member comprises two or more leg units in contact with the inner wall surface of the column and the porous filter.

3. The porous filter column according to claim 1,
wherein a contact surface of the leg unit and the inner wall surface of the column have a circular arc shape.

4. The porous filter column according to claim 3,
wherein a curvature of the circular arc shape of the contact surface of the leg unit is the same as a curvature of the circular arc shape of the inner wall surface of the column.

5. The porous filter column according to claim 4,
wherein a central angle of the circular arc shape is from 10° to 30°.

6. The porous filter column according to claim 1,
wherein the hollow unit of the hollow member is circular.

7. The porous filter column according to claim 1,
wherein the porous filter has a nucleic acid adsorptivity.

8. A reagent cartridge for accommodating a liquid to separate and purify a nucleic acid from an analyte, and dispensing the liquid using a dispensing tip, comprising:
an analyte accommodating unit which accommodates the analyte,
a liquid accommodating unit which accommodates the liquid,
a liquid waste accommodating unit which accommodates a liquid waste generated in the separation and purification, and
a porous filter column which purifies the nucleic acid of the analyte
wherein the porous filter column is the porous filter column according to claim 1.

9. A nucleic acid purification kit, comprising:
the reagent cartridge according to claim 8, and
a dispensing tip accommodator for accommodating a plurality of dispensing tips.
